(19) United States
(12) Patent Application Publication
(10) Pub. No.: US 2026/0297261 A1
(43) Pub. Date: Oct. 1, 2026

(54) POLYIMIDE-BASED RESIN PRECURSOR, PHOTOSENSITIVE RESIN COMPOSITION, METHOD FOR PRODUCING RESIN FILM, AND AMIC ACID ESTER COMPOUND

(71) Applicant: Resonac Corporation, Tokyo (JP)

(72) Inventors: Shintaro NAGAYAMA, Tokyo (JP); Yohei ISHIKAWA, Tokyo (JP); Mariko IWATA, Tokyo (JP)

(73) Assignee: Resonac Corporation, Tokyo (JP)

(21) Appl. No.: 19/478,344

(22) PCT Filed: Jul. 7, 2023
(86) PCT No.: PCT/JP2023/025333

§ 371 Date: Oct. 24, 2025

(51) Int. Cl. C08G 73/12; C07C 233/07; G03F 7/40

(52) U.S. Cl. CPC C08G 73/12; C07C 233/07; G03F 7/40

(57) ABSTRACT

A polyimide-based resin precursor includes at least one of a structural unit represented by the following formula (A1):

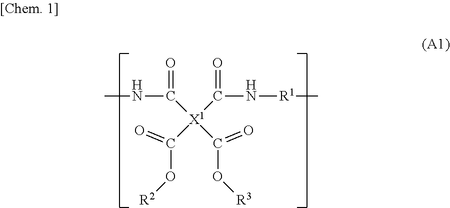

or a structural unit represented by formula (A2):

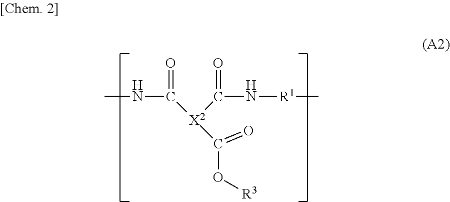

At least one of R² or R³ is an optionally substituted aromatic group, and at least one of R² or R³ is a group including a photopolymerizable group. R⁴ represents an aromatic group substituted with a group including a photopolymerizable group.

### TECHNICAL FIELD

The present disclosure relates to a polyimide-based resin precursor, a photosensitive resin composition, a method for producing a resin film, and an amic ester compound.

### BACKGROUND ART

An insulating resin layer constituting a semiconductor device may be formed from a resin composition including a polyimide precursor that forms a polyimide upon heating (for example, see Patent Literature 1).

### CITATION LIST

### Patent Literature

[Patent Literature 1] Japanese Unexamined Patent Publication No. 2021-196482

### SUMMARY OF INVENTION

### Technical Problem

An aspect of the present disclosure relates to a polyimide-based resin precursor that allows for the formation of a polyimide-based resin including a structural unit having an imide group by heating at a low temperature.

### Solution to Problem

The present disclosure includes the following.
[1] A polyimide-based resin precursor, comprising at least one of a structural unit represented by the following formula (A1):

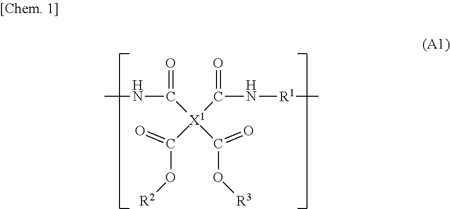

or a structural unit represented by formula (A2):

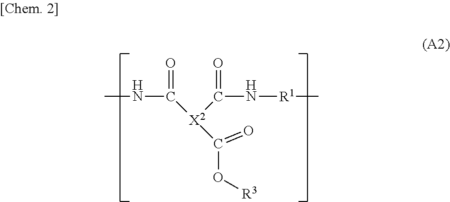

wherein in formula (A1), X¹ represents a tetravalent organic group, R¹ represents a divalent organic group, R² and R³ each independently represent a monovalent organic group, at least one of R² or R³ is an optionally substituted aromatic group, and at least one of R² or R³ is a group including a photopolymerizable group, and
wherein in formula (A2), X² represents a trivalent organic group, R¹ represents a divalent organic group, and R⁴ represents an aromatic group substituted with a group including a photopolymerizable group.
[2] A photosensitive resin composition comprising the polyimide-based resin precursor according to [1].
[3] A method for producing a resin film, comprising:
irradiating a portion of a photosensitive resin film comprising the polyimide-based resin precursor according to [1] with actinic radiation;
forming a patterned film by removing a portion of the photosensitive resin film; and
forming a resin film including an imide-based resin including a structural unit having an imide group by heating the patterned film.
[4] The method according to [3], wherein the resin film is formed by heating the patterned film to 200° C. or lower.
[5] An amic ester compound represented by the following formula (I):

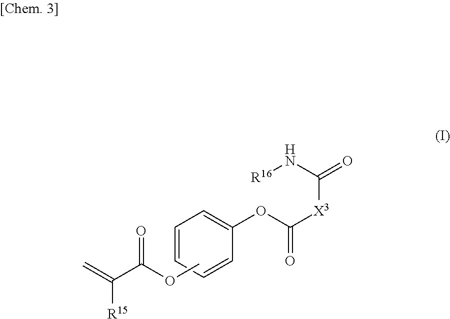

wherein R¹⁵ represents a hydrogen atom or a methyl group, R¹⁶ represents an alkyl group or an aryl group, and X³ represents a methanediyl group, an ethane-1,2-diyl group, an ethene-1,2-diyl group, or a 1,2-phenylene group.

### Advantageous Effects of Invention

A polyimide-based resin precursor is provided that allows for the formation of a polyimide-based resin including a structural unit having an imide group by heating at a low temperature. By heating a resin film including the polyimide-based resin precursor at a low temperature, a resin film with less coloration can be formed.

### BRIEF DESCRIPTION OF DRAWINGS

FIG. 1 is a process diagram illustrating an example of a method for producing a resin film.

FIG. 2 is a DSC curve of an amic ester compound.

FIG. 3 is a ¹H NMR spectrum of an amic ester compound.

FIG. 4 is a ¹H NMR spectrum of an amic ester compound.

### DESCRIPTION OF EMBODIMENTS

The present invention is not limited to the following examples.

An example of the polyimide-based resin precursor according to the present disclosure is a polymer including at least one of a structural unit represented by the following formula (A1) or a structural unit represented by the following formula (A2).

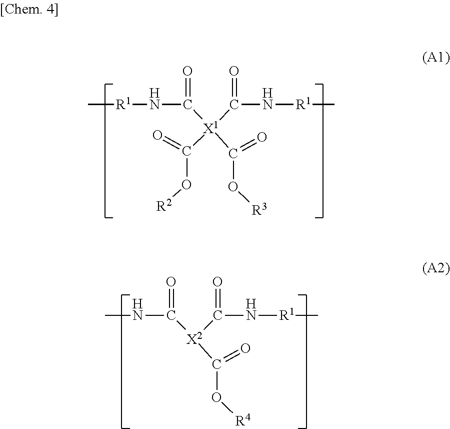

In formula (A1), X¹ represents a tetravalent organic group, R¹ represents a divalent organic group, and R² and R³ each independently represent a monovalent organic group. At least one of R² or R³ is an optionally substituted aromatic group. Further, at least one of R² or R³ is a group including a photopolymerizable group. One or both of R² or R³ may be an aromatic group substituted with a group including a photopolymerizable group. Alternatively, one of R² or R³ may be an aromatic group not substituted with a group including a photopolymerizable group, and the other of R² or R³ may be a group including a photopolymerizable group and other than an aromatic group. In a plurality of structural units represented by formula (A1) included in the polyimide-based resin precursor, the combinations of R¹, R², R³, and X¹ may be the same as or different from each other.

In formula (A2), X² represents a trivalent organic group, R¹ represents a divalent organic group, and R⁴ represents an aromatic group substituted with a group including a photopolymerizable group. In a plurality of structural units represented by formula (A2) included in the polyimide-based resin precursor, the combinations of R¹, R⁴, and X² may be the same as or different from each other.

Imidization proceeds by the reaction between the amide group and the carboxylic acid ester group in the structural unit represented by formula (A1) or (A2), thereby forming a polyimide-based resin. A polyimide-based resin formed from a polyimide-based resin precursor mainly composed of the structural unit represented by formula (A1) may be referred to as a polyimide resin. A polyimide-based resin formed from a polyimide-based resin precursor mainly composed of the structural unit represented by formula (A2) may be referred to as a polyamide-imide resin.

The structural unit represented by formula (A1) can form, for example, a structural unit represented by the following formula (B1) by eliminating an alcohol compound (R²—OH, R³—OH) through imidization. The structural unit represented by formula (A2) can form, for example, a structural unit represented by the following formula (B2) by eliminating an alcohol compound (R⁴—OH) through imidization. The polyimide-based resin formed by imidization may include at least one of a structural unit represented by formula (B1) or a structural unit represented by formula (B2).

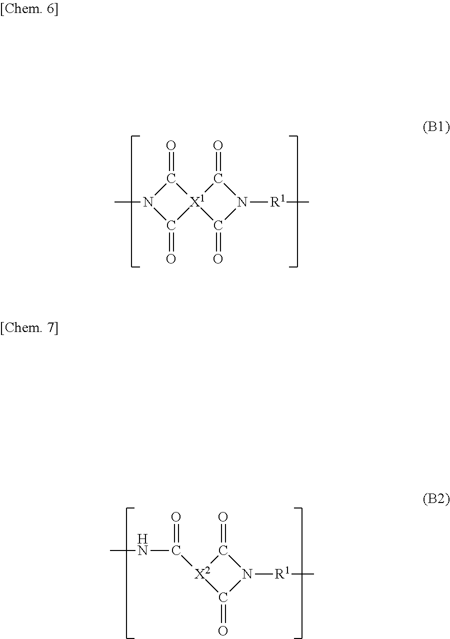

When at least one of R² or R³ is an optionally substituted aromatic group and R⁴ is an optionally substituted aromatic group, an aromatic alcohol compound is eliminated by imidization. As confirmed in the verification tests described later, the imidization reaction in which an aromatic alcohol compound having a phenolic hydroxyl group with relatively high acidity is eliminated proceeds efficiently at a relatively low temperature. Therefore, the polyimide-based resin precursor according to the present disclosure allows for the formation of a polyimide-based resin including a structural unit having an imide group by heating at a lower temperature.

The molecular weight of the aromatic alcohol compound eliminated by imidization may be 50 or more and 100,000 or less. At least one of R² or R³, and R⁴ can be a residue of an aromatic alcohol compound having a molecular weight within these ranges.

Examples of the aromatic group as R², R³, or R⁴ include a monocyclic aromatic group such as a phenyl group, and a polycyclic aromatic group such as a naphthyl group. The aromatic group as R², R³, or R⁴ may be substituted. The aromatic group as R², R³, or R⁴ may be substituted with a group including a photopolymerizable group (for example, a group represented by formula (10) described later), or may be substituted with a substituent not including a photopolymerizable group. Examples of the substituent not including a photopolymerizable group include an alkyl group, a halogeno group, a halogenated alkyl group, an aryl group (for example, a phenyl group), a halogenated aryl group, an alkylaryl group, a halogenated alkylaryl group, an alkoxy group, an aryloxy group, an alkoxycarbonyl group, an aryloxycarbonyl group, an alkylcarbonyloxy group, and an arylcarbonyloxy group.

The photopolymerizable group included in R², R³, or R⁴ is a functional group having a double bond, and may be, for example, a methacryloyl group, an acryloyl group, an acrylamide group, an allyl group, a vinyl group, a styryl group, or a combination thereof. The group including a photopolymerizable group can be, for example, a group represented by the following formula (10). In formula (10), R⁵, R⁶, and R⁷ each independently represent a hydrogen atom or an alkyl group having 1 to 3 carbon atoms, and n represents an integer of 0 to 10. R⁵ may be a hydrogen atom or a methyl group. R⁶ and R⁷ may be hydrogen atoms. n may be 0. One or both of R² or R³ may be an aromatic group substituted with a group represented by formula (10). R⁴ may be an aromatic group substituted with a group represented by formula (10).

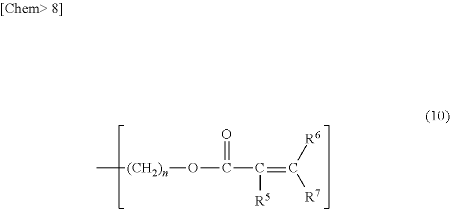

One of R² or R³ may be an aromatic group which may be substituted with a group not including a photopolymerizable group, and the other of R² or R³ may be a group represented by formula (10). In that case, n in formula (10) is an integer of 1 to 10.

One of R² or R³ may be an aromatic group substituted with a group including a photopolymerizable group, and the other of R² or R³ may be a hydrogen atom or an optionally substituted aliphatic group (for example, an alkyl group having 1 to 40 carbon atoms).

X¹ in formula (A1) can be an aromatic group having a plurality of carbon atoms, including two first carbon atoms bonded to a —COOR² group or a —COOR³ group, and second carbon atoms bonded to an amide group, which are adjacent to the respective first carbon atoms or adjacent to carbon atoms adjacent to the first carbon atoms. The first carbon atom and the second carbon atom adjacent to each other are directly bonded by a covalent bond. X¹ may be a tetravalent organic group having 6 to 40 carbon atoms. The tetravalent organic group represented by X¹ may include an optionally substituted aromatic group, an optionally substituted cyclic aliphatic group, or a combination thereof.

X¹ in formula (A1) may be a tetravalent group represented by the following formula (a1), (a2), (a3), or (a4). These tetravalent groups are usually bonded to a carboxylic acid ester group (—COOR² group or —COOR³ group) and an amide group at the positions of the two bonding hands on both sides.

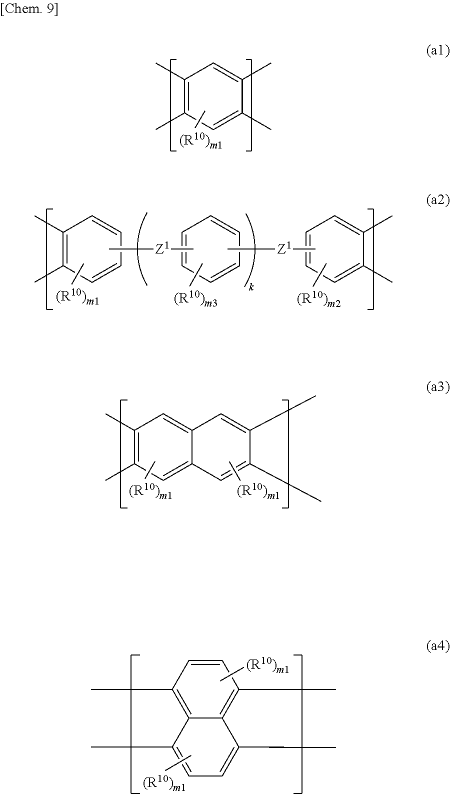

In formulas (a1) to (a4), R¹⁰ represents a fluorine atom, a hydrocarbon group having 1 to 10 carbon atoms, or a fluorinated hydrocarbon group having 1 to 10 carbon atoms, m1 represents an integer of 0 to 2, m2 represents an integer of 0 to 3, and m3 represents an integer of 0 to 4. A plurality of R¹⁰s, m1s, and m2s in one tetravalent group may be the same as or different from each other, respectively. In formula (a2), Z¹ represents a direct bond, a methanediyl group, a propane-2,2-diyl group, a 1,1,1,3,3,3-hexafluoropropane-2,2-diyl group, a carbonyl group, a sulfonyl group, a thio group, a carbonyloxy group, an oxy group, or a fluorene-9,9-diyl group, and k represents an integer of 0 to 2. A plurality of Z¹s in one tetravalent group may be the same as or different from each other. R¹⁰ may be a fluorine atom, an alkyl group having 1 to 10 carbon atoms (for example, a methyl group), or a fluorinated alkyl group having 1 to 10 carbon atoms (for example, a trifluoromethyl group). m1, m2, and m3 may each be 0.

X² in formula (A2) can be an aromatic group having a plurality of carbon atoms, including one first carbon atom bonded to a —COOR⁴ group, and a second carbon atom bonded to an amide group, which is adjacent to the first carbon atom or adjacent to a carbon atom adjacent to the first carbon atom. The first carbon atom and the second carbon atom adjacent to each other are directly bonded by a covalent bond. X² may be a trivalent organic group having 6 to 40 carbon atoms. The trivalent organic group represented by X² may include an optionally substituted aromatic group, an optionally substituted cyclic aliphatic group, or a combination thereof.

X² in formula (A2) may be a trivalent group represented by the following formula (a5), (a6), (a7), or (a8). R¹⁰, m1, m2, and m3 in formulas (a5) to (a8) are defined in the same manner as R¹⁰, m1, m2, and m3 in formulas (a1) to (a4). A plurality of R¹⁰s and m3s in one trivalent group may be the same as or different from each other, respectively. These trivalent groups are usually bonded to an amide group at the position of one bonding hand on one side, and bonded to a carboxylic acid ester group (—COOR⁴ group) and an amide group at the positions of two bonding hands on the other side.

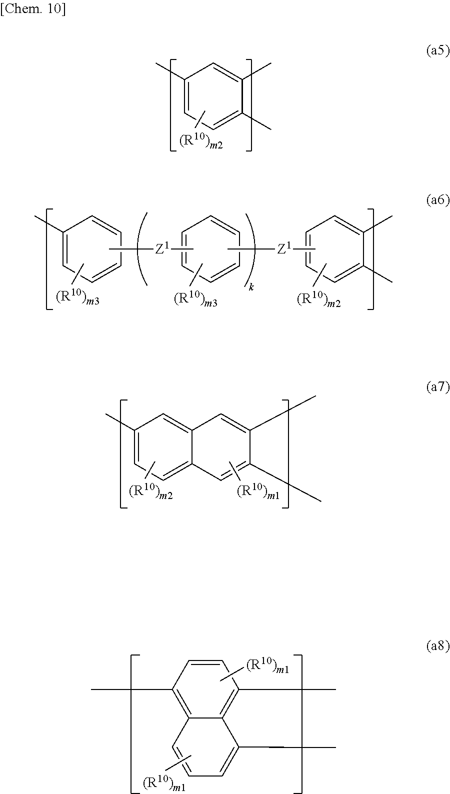

R¹ in formulas (A1) and (A2) may be a divalent organic group having 6 to 40 carbon atoms. R¹ may include an optionally substituted aromatic group, an optionally substituted cyclic aliphatic group, a siloxane group, or a combination thereof. R¹ in formulas (A1) and (A2) may be a divalent group represented by the following formula (a10), (a11), or (a12).

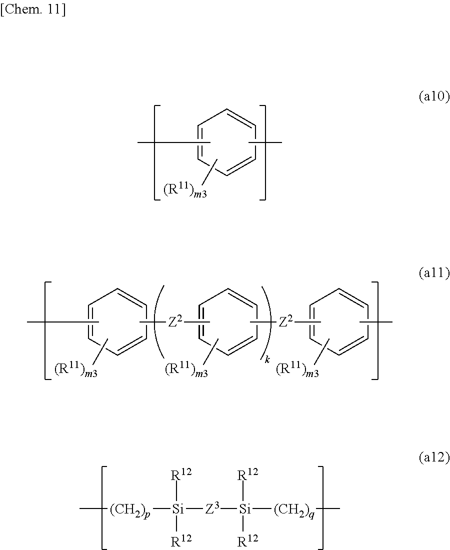

In formulas (a10) and (a11), R¹¹ represents a fluorine atom, a hydrocarbon group having 1 to 10 carbon atoms, or a fluorinated hydrocarbon group having 1 to 10 carbon atoms, and m3 represents an integer of 0 to 4. A plurality of R¹¹s and m3s in one divalent group may be the same as or different from each other, respectively. m3 may be 0.

In formula (a11), Z² represents a direct bond, a methanediyl group, a propane-2,2-diyl group, a 1,1,1,3,3,3-hexafluoropropane-2,2-diyl group, a carbonyl group, a sulfonyl group, a thio group, a carbonyloxy group, an oxy group, a fluorene-9,9-diyl group, or an amide group, and k represents an integer of 0 to 2. A plurality of Z²s in one divalent group may be the same as or different from each other. R¹¹ may be a fluorine atom, an alkyl group having 1 to 10 carbon atoms (for example, a methyl group), or a fluorinated alkyl group having 1 to 10 carbon atoms (for example, a trifluoromethyl group).

In formula (a12), Z³ represents an oxy group or an arylene group (for example, a phenylene group), R¹² represents an alkyl group having 1 to 10 carbon atoms (for example, a methyl group), and p and q each independently represent an integer of 1 to 10. A plurality of R¹²s in one divalent group may be the same as or different from each other. p and q may be an integer of 1 to 3.

The polyimide-based resin precursor may be a polymer including only the structural unit represented by formula (A1) or (A2) as a repeating unit, or may be a polymer further including a structural unit other than these. For example, the polyimide-based resin precursor may further include a structural unit represented by the following formula (A1′) or (A2′). R¹, X¹, and X² in formulas (A1′) and (A2′) are defined in the same manner as R¹, X¹, and X² in formulas (A1) and (A2).

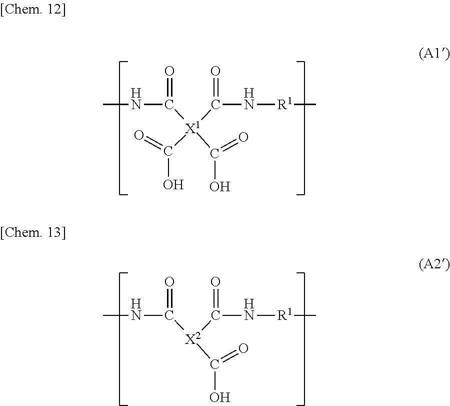

In the polyimide-based resin precursor, the proportion of the structural unit represented by formula (A1) or (A2) may be, for example, 50% by mass or more and 100% by mass or less, based on the mass of the polyimide-based resin precursor. In the polyimide-based resin precursor, the proportion of the structural unit represented by formula (A1) or (A2) may be 55% by mass or more, 60% by mass or more, 65% by mass or more, 70% by mass or more, 75% by mass or more, 80% by mass or more, 85% by mass or more, 90% by mass or more, 95% by mass or more, 96% by mass or more, 97% by mass or more, 98% by mass or more, or 99% by mass or more, and may be 99% by mass or less, 98% by mass or less, 97% by mass or less, 96% by mass or less, 95% by mass or less, or 90% by mass or less, based on the mass of the polyimide-based resin precursor.

The weight-average molecular weight of the polyimide-based resin precursor may be 8,000 or more and 150,000 or less, 9,000 or more and 50,000 or less, or 18,000 or more and 40,000 or less. The weight-average molecular weight here can be a value in terms of standard polystyrene, measured by gel permeation chromatography.

A polyimide-based resin precursor (polyimide resin precursor) having the structural unit represented by formula (A1) can be produced by a method including, for example, forming a polyamic acid having a carboxy group by polycondensation of a tetracarboxylic dianhydride and a diamine compound (NH₂—R¹—NH₂), and converting at least a part of the carboxy groups in the polyamic acid into carboxylic acid ester groups by reaction of the polyamic acid with an alcohol compound (R²—OH, R³—OH) including an aromatic alcohol compound. In the polyimide-based resin precursor obtained by this method, X¹ is usually a residue derived from the tetracarboxylic dianhydride, and R¹ is a residue derived from the diamine compound.

A polyimide-based resin precursor (polyamide-imide resin precursor) having the structural unit represented by formula (A2) can be produced by a method including, for example, forming a polyamic acid having a carboxy group by polyaddition of a tricarboxylic anhydride and a diamine compound (NH₂—R¹—NH₂), and converting at least a part of the carboxy groups in the polyamic acid into carboxylic acid ester groups by reaction of the polyamic acid with an alcohol compound including an aromatic alcohol compound (R⁴—OH). In the polyimide-based resin precursor obtained by this method, X² is usually a residue derived from the tricarboxylic anhydride, and R¹ is a residue derived from the diamine compound.

By using both a tetracarboxylic dianhydride and a tricarboxylic anhydride as raw materials, it is also possible to obtain a polyimide-based resin precursor having both the structural unit represented by formula (A1) and the structural unit represented by formula (A2).

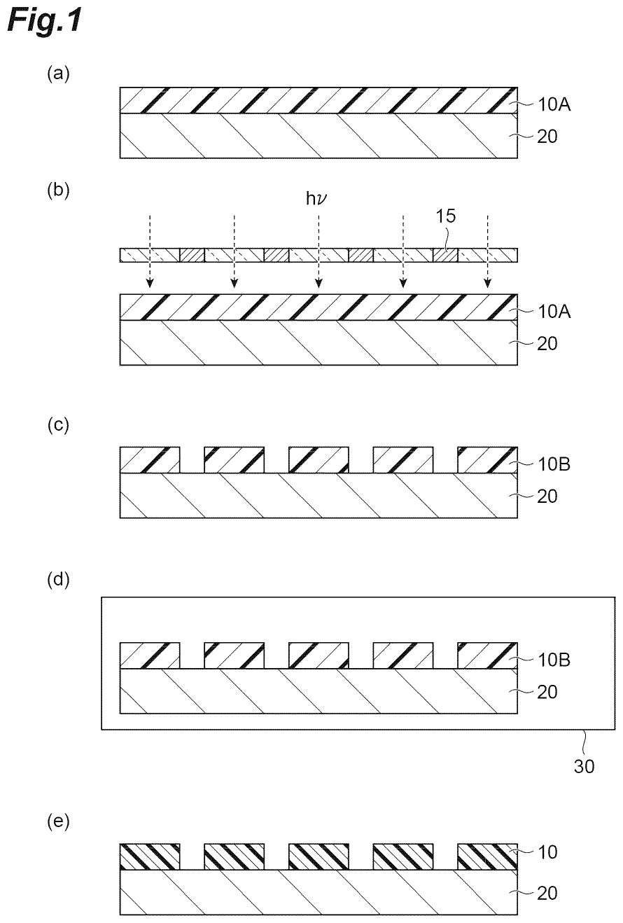

Using the photosensitive resin composition including the polyimide-based resin precursor, for example, a patterned resin film can be formed. FIG. 1 is a process diagram illustrating an example of a method for producing a patterned resin film. The method shown in FIG. 1 includes forming a photosensitive resin film 10A including the photosensitive resin composition on a substrate 20, irradiating a portion of the photosensitive resin film 10A with actinic radiation hv (for example, ultraviolet light), forming a patterned resin film 10B by removing a portion of the photosensitive resin film 10A, and forming a resin film 10 including an imide-based resin including a structural unit having an imide group by heating the resin film 10B.

The photosensitive resin film 10A can be formed by applying the photosensitive resin composition onto the substrate 20 and, if necessary, removing the solvent from the coating film. By irradiating with actinic radiation hv through a mask 15 having a light-transmitting portion, a portion of the photosensitive resin film 10A is irradiated with the actinic radiation hv. Thereafter, the patterned resin film 10B is formed by a normal development technique. The resin film 10 including the polyimide-based resin is formed by heating the resin film 10B with a heating device 30 to advance imidization. The heating device 30 can be, for example, a heating furnace.

Imidization can proceed by heating the resin film 10B at a relatively low temperature, and the resin film 10 including the polyimide-based resin can be formed. The heating temperature for imidization may be, for example, 200° C. or lower, 190° C. or lower, 180° C. or lower, or 170° C. or lower, and may be 140° C. or higher, or 150° C. or higher. The heating time for imidization may be, for example, 30 minutes or more and 180 minutes or less.

The photosensitive resin composition may include other components in addition to the polyimide-based resin precursor, if necessary. For example, the photosensitive resin composition may include a solvent that dissolves or disperses the polyimide-based resin precursor, and may further include a photopolymerization initiator and/or a photopolymerizable monomer.

The content of the polyimide-based resin precursor in the photosensitive resin composition may be 50% by mass or more and 100% by mass or less, based on the total mass of the components of the photosensitive resin composition other than the solvent.

The solvent may be a polar organic solvent. Examples of the solvent include 3-methoxy-N,N-dimethylpropionamide, N,N-dimethylformamide, N-methyl-2-pyrrolidone, N-ethyl-2-pyrrolidone, N,N-dimethylacetamide, dimethyl sulfoxide, diethylene glycol dimethyl ether, cyclopentanone, γ-butyrolactone, α-acetyl-y-butyrolactone, tetramethylurea, 1,3-dimethyl-2-imidazolidinone, N-cyclohexyl-2-pyrrolidone, and 2-octanone. These can be used alone or in a combination of two or more.

The content of the solvent may be 30 parts by mass or more and 1500 parts by mass or less, 100 parts by mass or more and 1000 parts by mass or less, or 100 parts by mass or more and 860 parts by mass or less, with respect to 100 parts by mass of the polyimide-based resin precursor.

The photopolymerization initiator may be a photoradical polymerization initiator, and examples thereof include benzophenone derivatives such as benzophenone, methyl o-benzoylbenzoate, 4-benzoyl-4′-methyldiphenylketone, dibenzylketone, and fluorenone; acetophenone derivatives such as 2,2′-diethoxyacetophenone, 2-hydroxy-2-methylpropiophenone, and 1-hydroxycyclohexyl phenyl ketone; thioxanthone derivatives such as thioxanthone, 2-methylthioxanthone, 2-isopropylthioxanthone, and diethylthioxanthone; benzyl derivatives such as benzyl, benzyl dimethyl ketal, and benzyl-β-methoxyethyl acetal; benzoin derivatives such as benzoin and benzoin methyl ether; oximes such as 1-phenyl-1,2-butanedione-2-(o-methoxycarbonyl)oxime, 1-phenyl-1,2-propanedione-2-(o-methoxycarbonyl) oxime, 1-phenyl-1,2-propanedione-2-(o-ethoxycarbonyl) oxime, 1-phenyl-1,2-propanedione-2-(o-benzoyl)oxime, 1,3-diphenylpropanetrione-2-(o-ethoxycarbonyl) oxime, and 1-phenyl-3-ethoxypropanetrione-2-(o-benzoyl) oxime; N-arylglycines such as N-phenylglycine; peroxides such as benzoyl perchloride; aromatic biimidazoles; and titanocenes. The photopolymerization initiator may be a photoacid generator, and an example thereof includes α-(n-octanesulfonyl-oxyimino)-4-methoxybenzyl cyanide.

The content of the photopolymerization initiator may be 0.1 parts by mass or more and 10 parts by mass or less, or 1 part by mass or more and 8 parts by mass or less, with respect to 100 parts by mass of the polyimide-based resin precursor.

The photosensitive resin composition may further include a photopolymerizable monomer. This photopolymerizable monomer is a compound having one or more photopolymerizable groups and is selected from compounds different from the polyimide precursor and additives. The photopolymerizable group of the photopolymerizable monomer may be, for example, a methacryloyl group or an acryloyl group. The photopolymerizable monomer may be one or more selected from acrylic acid esters of alcohol compounds, methacrylic acid esters of alcohol compounds, acrylamide and its derivatives, and methacrylamide and its derivatives. The acrylic acid ester can be a monoacrylate or a polyacrylate (for example, diacrylate, triacrylate, tetraacrylate). The methacrylic acid ester can be a monomethacrylate or a polymethacrylate (for example, dimethacrylate, trimethacrylate, tetramethacrylate).

Examples of the alcohol compound that forms the acrylic acid ester or methacrylic acid ester include alkyl alcohols, alkane polyols, polyether polyols, aromatic polyols, and ethylene oxide adducts or propylene oxide adducts of these alcohol compounds. An example of the alkyl alcohol includes isoborneol. Examples of the alkane polyol include ethylene glycol, propylene glycol, neopentyl glycol, glycerol, 1,4-butanediol, 1,6-hexanediol, cyclohexanediol, trimethylolpropane, and pentaerythritol. Examples of the polyether polyol include polyethylene glycol (for example, diethylene glycol, tetraethylene glycol) and polypropylene glycol. Examples of the aromatic polyol include bisphenol A and trihydroxybenzene.

The content of the photopolymerizable monomer in the photosensitive resin composition may be 1 part by mass or more and 50 parts by mass or less with respect to 100 parts by mass of the polyimide-based resin precursor. The content of the photopolymerizable monomer may be 5 parts by mass or more, 10 parts by mass or more, 15 parts by mass or more, 20 parts by mass or more, 25 parts by mass or more, or 30 parts by mass or more, and may be 45 parts by mass or less, or 40 parts by mass or less, with respect to 100 parts by mass of the polyimide-based resin precursor.

An aspect of the present disclosure relates to an amic ester compound represented by the following formula (I). Since the amic ester compound of formula (I) can form an imide by the reaction between the amide group and the carboxylic acid ester group bonded via X³, it can be used to verify the reactivity of the imidization of the polyimide-based resin precursor. This compound can also be used as a photopolymerizable monomer.

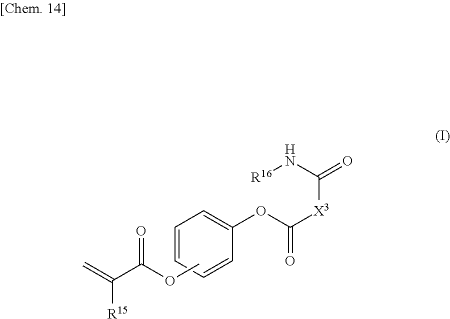

In formula (I), R¹⁵ represents a hydrogen atom or a methyl group, R¹⁶ represents an alkyl group or an aryl group (for example, a phenyl group), and X³ represents a methanediyl group, an ethane-1,2-diyl group, an ethene-1,2-diyl group, or a 1,2-phenylene group. The (meth)acryloyloxy group (CH₂═C(R¹⁵)COO—) may be bonded to a carbon atom at the para-position with respect to the group including X³. Verification Tests

### 1. Synthesis of Amic Ester Compounds

### Amic Ester Compound Ia

Succinic anhydride 1 (50.0 mmol) was reacted with aniline 2 (50.0 mmol) in THF (30 mL) at 25° C. to obtain an amic acid compound 3. The amic acid compound 3 (2.5 mmol) was reacted with 4-hydroxyphenyl methacrylate 4a (2.5 mmol) in THF (solvent) in the presence of EDCI (1-(3-dimethylaminopropyl)-3-ethylcarbodiimide hydrochloride, 3 mol) and BHT (dibutylhydroxytoluene, 1000 ppm) while raising the temperature from 0° C. to 25° C. to produce an amic ester compound 1a. Crystals of purified amic ester compound 1a (2.25 mmol) were obtained by recrystallization from a mixed solvent of hexane and acetone (mass ratio hexane:acetone=95:5).

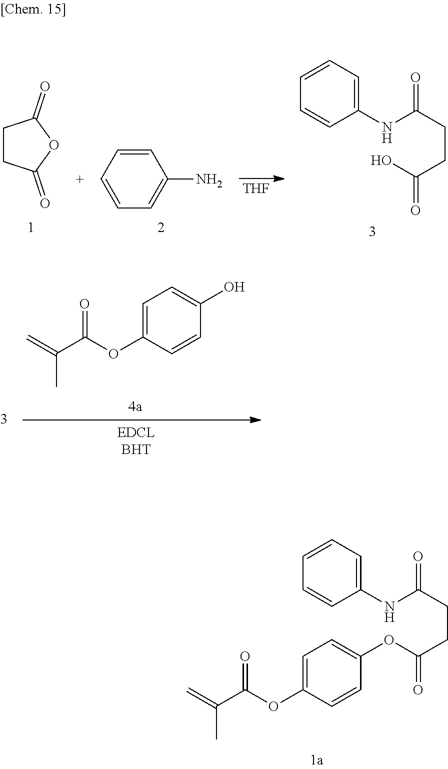

Amic Ester Compound II (Comparative Compound) Succinic anhydride 1 (25.0 mmol) was reacted with 2-hydroxyethyl methacrylate 4b (25.0 mmol) in THF (20 mL) at 25° C. in the presence of triethylamine (150 mmol) and BHT (dibutylhydroxytoluene, 1000 ppm) to obtain a dicarboxylic acid monoester compound 5. The dicarboxylic acid monoester compound 5 (2.0 mmol) was reacted with aniline 2 (2.0 mmol) in THF (5.0 mL) in the presence of EDCI (1-(3-dimethylaminopropyl)-3-ethylcarbodiimide hydrochloride, 2.4 mmol) while raising the temperature from 0° C. to 25° C. to produce an amic ester compound II. Crystals of purified amic ester compound II (1.90 mmol) were obtained by recrystallization from a mixed solvent of hexane and acetone (mass ratio hexane:acetone=95:5).

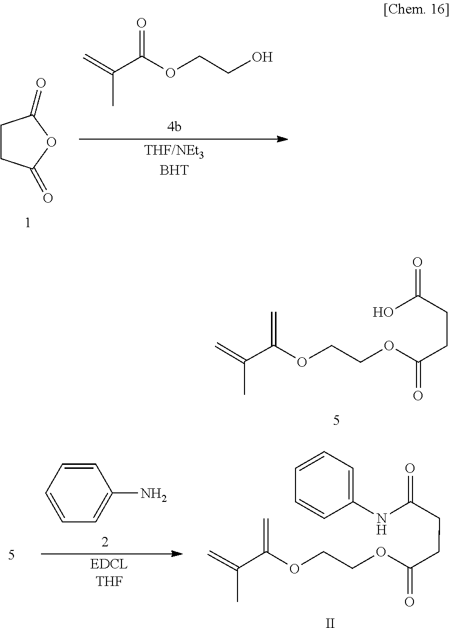

2. Differential Scanning calorimetry (DSC)

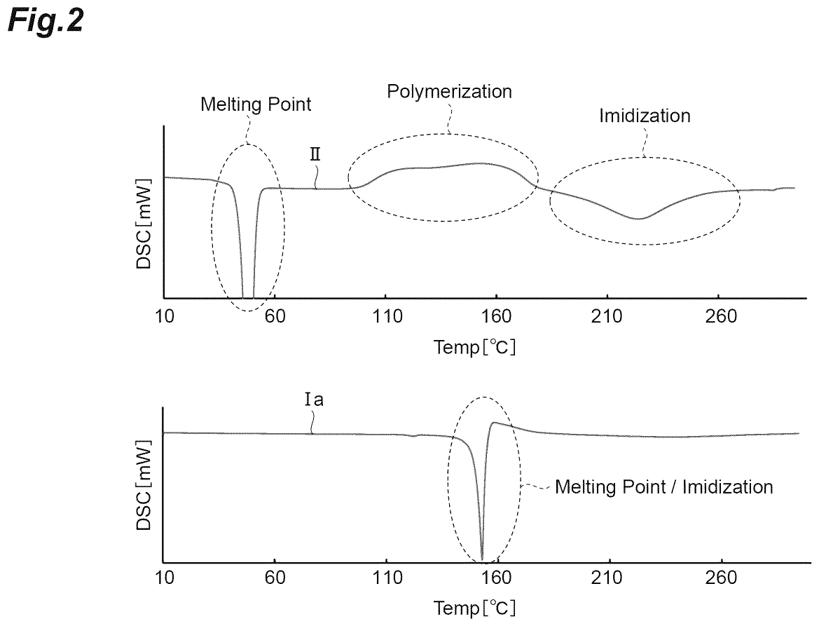

Differential scanning calorimetry (heating rate: 10° C./min) was performed on the amic ester compounds Ia and II. FIG. 2 is a DSC curve showing the relationship between heat flow and temperature. In the case of the amic ester compound II, endothermic heat due to imidization was observed mainly in the region of 200° C. or higher. The amic ester compound Ia showed a melting point and endothermic heat associated with imidization at a low temperature of 160° C. or lower.

### 3. Reaction Test

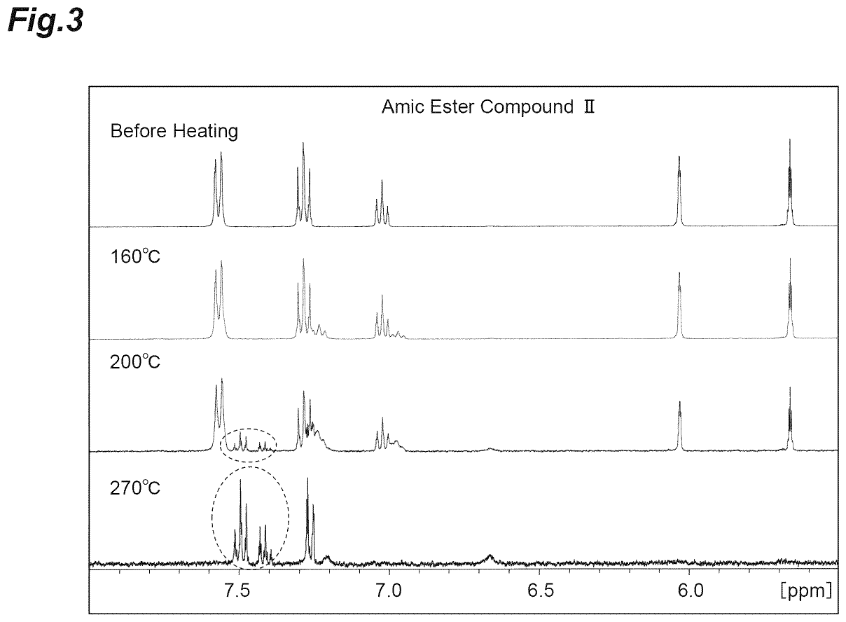

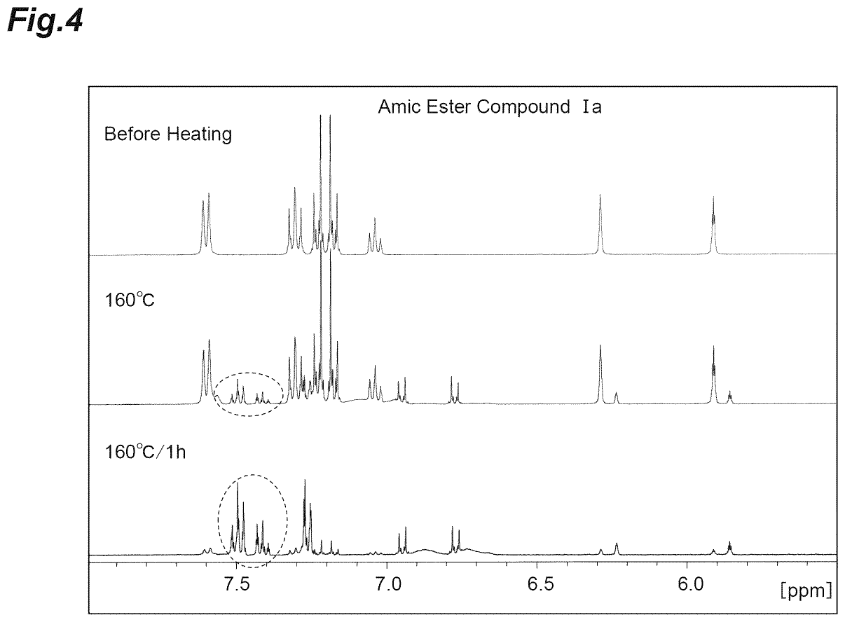

Crystals of the amic ester compound II were heated from 10° C. to 270° C. at a heating rate of 10° C./min. ¹H NMR spectra (solvent:deuterated dimethyl sulfoxide) of the crystals were measured before heating, and at the time points of 160° C., 200° C., or 270° C. Crystals of the amic ester compound Ia were heated from 10° C. to 160° C. at a heating rate of 10° C./min, and subsequently heated at 160° C. for 1 hour. ¹H NMR spectra (solvent:deuterated dimethyl sulfoxide) of the crystals were measured before heating, at the time point of 160° C., and after heating at 160° C. for 1 hour. FIG. 3 and FIG. 4 are ¹H NMR spectra of the amic ester compounds II and Ia, respectively. In the case of the amic ester compound II, no signal attributed to an imide group was observed at 160° C., and a signal of an imide group was observed in the region of 7.4-7.5 ppm at 200° C. or higher. In the case of the amic ester compound Ia, a signal of an imide group was observed at the time point of 160° C., and it was confirmed that the imidization reaction proceeded sufficiently by heating at 160° C. for 1 hour.

From the above verification tests, it was suggested that by introducing an ester group of an aromatic alcohol into the structure of the amic ester of the polyimide-based resin precursor, a polyimide-based resin can be formed at a lower temperature.

### REFERENCE SIGNS LIST

10A . . . photosensitive resin film, 10 . . . resin film including polyimide-based resin, 20 . . . substrate.

1. A polyimide-based resin precursor, comprising at least one of a structural unit represented by the following formula (A1): 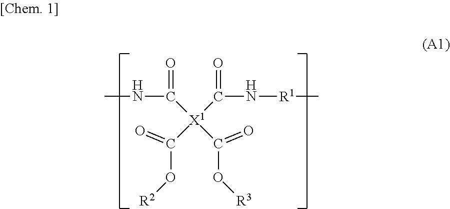
or a structural unit represented by the following formula (A2): 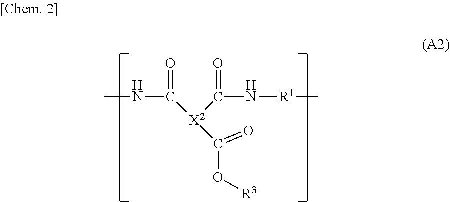
wherein in formula (A1), X¹ represents a tetravalent organic group, R¹ represents a divalent organic group, R² and R³ each independently represent a monovalent organic group, at least one of R² or R³ is an optionally substituted aromatic group, and at least one of R² or R³ is a group including a photopolymerizable group, and
wherein in formula (A2), X² represents a trivalent organic group, R¹ represents a divalent organic group, and R⁴ represents an aromatic group substituted with a group including a photopolymerizable group.

2. A photosensitive resin composition comprising the polyimide-based resin precursor according to claim 1.

3. A method for producing a resin film, comprising:
irradiating a portion of a photosensitive resin film comprising the polyimide-based resin precursor according to claim 1 with actinic radiation;
forming a patterned film by removing a portion of the photosensitive resin film; and
forming a resin film comprising an imide-based resin comprising a structural unit having an imide group by heating the patterned film.

4. The method according to claim 3, wherein the resin film is formed by heating the patterned film to 200° C. or lower.

5. An amic ester compound represented by the following formula (I): 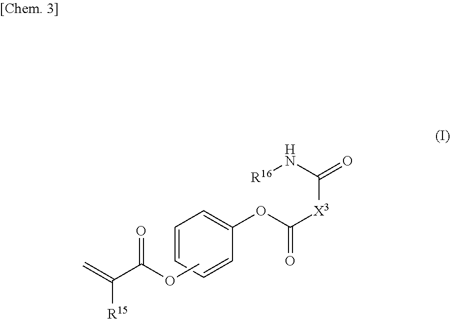
wherein R¹⁵ represents a hydrogen atom or a methyl group, R¹⁶ represents an alkyl group or an aryl group, and X³ represents a methanediyl group, an ethane-1,2-diyl group, an ethene-1,2-diyl group, or a 1,2-phenylene group.
